(19) United States
(12) Patent Application Publication
(10) Pub. No.: US 2013/0110222 A1
(43) Pub. Date: May 2, 2013

(54) MEDICAL DEVICES INCLUDING SUPERHYDROPHOBIC OR SUPEROLEOPHOBIC SURFACES

(71) Applicant: Joram Slager, St. Louis Park, MN (US)

(72) Inventor: Joram Slager, St. Louis Park, MN (US)

(21) Appl. No.: 13/588,118

(22) Filed: Aug. 17, 2012

Related U.S. Application Data
(60) Provisional application No. 61/525,491, filed on Aug. 19, 2011.

(51) Int. Cl. A61F 2/95

(52) U.S. Cl. CPC A61F 2/95
USPC 623/1.11

(57) ABSTRACT

The present invention relates to medical devices including a superhydrophobic surface or coating, a superoleophobic surface or coating, a coating or surface that is both superhydrophobic and superoleophobic, or a combination of such coatings and surfaces. Such a coating or surface can impart advantageous lubricity, hemocompatibility, or both to the medical device or its surface.

### CROSS-REFERENCE TO RELATED APPLICATION

This application claims benefit of U.S. Provisional Application No. 61/525,491, filed Aug. 19, 2011, which application is incorporated herein by reference.

### FIELD OF THE INVENTION

The present invention relates to medical devices including a superhydrophobic surface or coating, a superoleophobic surface or coating, a coating or surface that is both superhydrophobic and superoleophobic, or a combination of such coatings and surfaces. Such a coating or surface can impart advantageous lubricity, hemocompatibility, or both to the medical device or its surface.

### BACKGROUND OF THE INVENTION

The chemical modification of surfaces to achieve desired chemical and/or physical characteristics has been previously described. Often, the various coatings and techniques referred to above are used to coat the surfaces of materials (e.g., medical devices) intended for temporary or permanent placement in the body. In turn, the resulting coatings typically provide a desired function or feature, such as lubricity, and must do so in a manner that provides the desired combination of such other properties as hemocompatibility, durability, and sterility.

There remains a need for improved hydrophobic, oleophobic, lubricious, or hemocompatible coatings for medical devices.

### SUMMARY OF THE INVENTION

The present invention relates to medical devices including a superhydrophobic surface or coating, a superoleophobic surface or coating, a coating or surface that is both superhydrophobic and superoleophobic, or a combination of such coatings and surfaces. Such a coating or surface can impart advantageous lubricity, hemocompatibility, or both to the medical device or its surface.

### BRIEF DESCRIPTION OF THE FIGURES

FIG. 1 schematically illustrates an embodiment of a medical device according to the present invention.

FIG. 2 schematically illustrates an embodiment of the medical device of FIG. 1.

FIG. 3 is a bar chart representing time @ half maximum clotting (sec) for Examples 1-7.

### DETAILED DESCRIPTION OF THE INVENTION

The present invention relates to a medical device that includes a surface that, for example, contacts a biological fluid or a surface of a medical device (either itself or another medical device). According to the present invention, the medical device can include a coating or surface that is superhydrophobic, superoleophobic, or both. The coating or surface can encompass all or part of the medical device. In an embodiment, the coating or surface imparts advantageous lubricity, hemocompatibility, or both to the medical device or its surface.

Suitable coatings, materials, or surfaces that are superhydrophobic, superoleophobic, or both include those described in U.S. patent application Ser. No. 12/538,632 (published as publication no. US 2010/0068434 A1), the disclosure of which is incorporated herein by reference. Additional such coatings are described in I. S. Bayer et al. Applied Physics Express 2 (2009) 125003 and in I. S. Bayer et al. Applied Surface Science 257 (2010) 823-826; the disclosures of which are incorporated herein by reference. Another example of suitable coatings, materials, or surfaces that are superhydrophobic, superoleophobic, or both is provided by U.S. Provisional Patent Application No. 61/434,217 (published as publication no. US ______ A1), the disclosure of which is incorporated herein by reference. Yet another example of suitable coatings, materials, or surfaces that are superhydrophobic, superoleophobic, or both is provided by L. Mischchenko et al. ACS Nano 4 (12), 7699-7707 (2010), the disclosure of which is incorporated herein by reference. Additional such surfaces, coatings or materials are described in greater detail hereinbelow.

Other publications that describe surfaces that are superhydrophobic, superoleophobic, or both include: “Bioinspired Self-Repairing Slippery Surfaces with Pressure-Stable Omniphobicity” Nature, Vol. 477; Sep. 22, 2011; 443-447 (Wong et al.) and “Liquid Infused Nanostructured Surfaces with Extreme Anti-Ice and Anti-Frost Performance” ACSNANO (2011) published on line as 10.1021/nn302310q (Kim et al.); both of which are incorporated herein by reference.

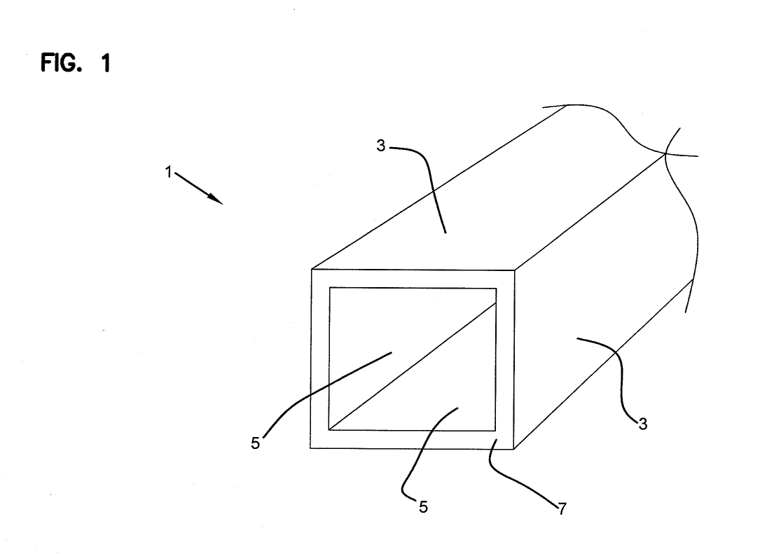

FIG. 1 schematically illustrates an embodiment of a medical device according to the present invention. Medical device 1 including one or more of outer surface 3, inner surface 5, and body 7. Although shown as a hollow rectangular solid, medical device 1 can have any of a variety of configurations. Medical device 1 can have a lumen or can be closed to its surroundings. In an embodiment, outer surface 3 is at least partially coated with or made from a substance that is superhydrophobic, superoleophobic, or both. In an embodiment, outer surface 3 is at least partially coated with or made from a plurality of substances that are superhydrophobic, superoleophobic, or both. In an embodiment, inner surface 5 is at least partially coated with or made from a substance that is superhydrophobic, superoleophobic, or both. In an embodiment, inner surface 5 is at least partially coated with or made from a plurality of substances that are superhydrophobic, superoleophobic, or both. In an embodiment, body 7 includes a substance that is superhydrophobic, superoleophobic, or both. In an embodiment, body 7 includes a plurality of substances that are superhydrophobic, superoleophobic, or both.

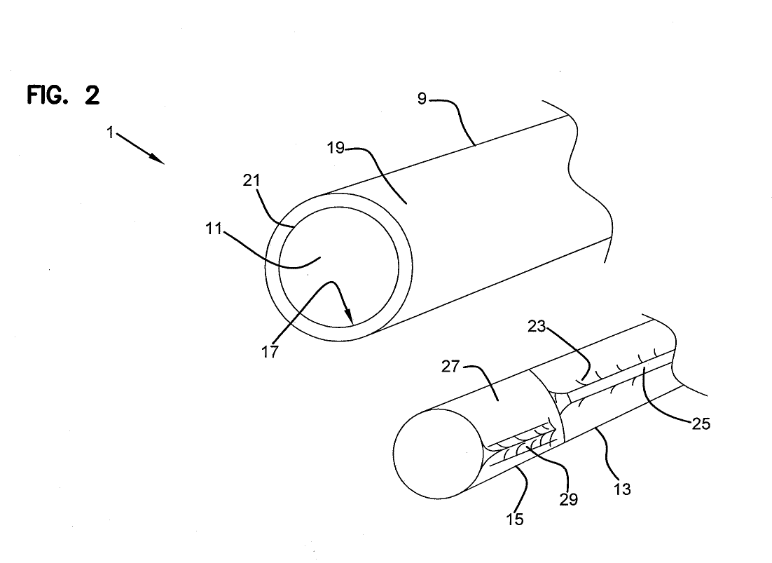

FIG. 2 schematically illustrates an embodiment of medical device 1. This embodiment is schematically illustrated as a tube (e.g., a catheter) 9 defining lumen 11. Although shown as a tube, this embodiment of the device can have any of a variety of configurations where one part of a device is configured to occupy a void in a second part of a device and they, for example, come into moveable contact with one another. Inner member 13 is configured to be at least partially disposed in lumen 11. Inner member 13 can be any of a variety of medically useful articles including a guide wire, a guide catheter, and the like. In an embodiment, inner member 13 includes implantable medical device 15. Implantable medical device 15 can be any of a variety of devices including, for example, a stent, a heart valve, or the like. Tube 9 can include body 21.

In an embodiment, inner surface 17 of tube 9 is at least partially coated with or made from a substance that is superhydrophobic, superoleophobic, or both. In an embodiment, inner surface 17 of tube 9 is at least partially coated with or made from a plurality of substances that are superhydrophobic, superoleophobic, or both. In an embodiment, outer surface 19 of tube 9 is at least partially coated with or made from a substance that is superhydrophobic, superoleophobic, or both. In an embodiment, outer surface 19 of tube 9 is at least partially coated with or made from a plurality of substances that are superhydrophobic, superoleophobic, or both. In an embodiment, body 21 of tube 9 is at least partially coated with or made from a substance that is superhydrophobic, superoleophobic, or both. In an embodiment, body 21 of tube 9 is at least partially coated with or made from a plurality of substances that are superhydrophobic, superoleophobic, or both.

In an embodiment, inner member 13 is at least partially coated with or made from a substance that is superhydrophobic, superoleophobic, or both. In an embodiment, inner member 13 is at least partially coated with or made from a plurality of substances that are superhydrophobic, superoleophobic, or both. In an embodiment, implantable medical device 15 is at least partially coated with or made from a substance that is superhydrophobic, superoleophobic, or both. In an embodiment, implantable medical device 15 is at least partially coated with or made from a plurality of substances that are superhydrophobic, superoleophobic, or both.

In an embodiment, inner member 13 includes outer surface 23. In an embodiment, outer surface 23 of inner member 13 is at least partially coated with or made from a substance that is superhydrophobic, superoleophobic, or both. In an embodiment, outer surface 23 of inner member 13 is at least partially coated with or made from a plurality of substances that are superhydrophobic, superoleophobic, or both.

In an embodiment, inner member 13 includes contact member 25, which protrudes from inner member 13 and is configured to contact inner surface 17 of tube 9. In an embodiment, contact member 25 is at least partially coated with or made from a substance that is superhydrophobic, superoleophobic, or both. In an embodiment, contact member 25 is at least partially coated with or made from a plurality of substances that are superhydrophobic, superoleophobic, or both.

In an embodiment, implantable medical device 15 includes outer surface 27. In an embodiment, outer surface 27 of implantable medical device 15 is at least partially coated with or made from a substance that is superhydrophobic, superoleophobic, or both. In an embodiment, outer surface 27 of implantable medical device 15 is at least partially coated with or made from a plurality of substances that are superhydrophobic, superoleophobic, or both.

In an embodiment, implantable medical device 15 includes contact portion 29, which protrudes from implantable medical device 15 and is configured to contact inner surface 17 of tube 9. In an embodiment, contact portion 29 of implantable medical device 15 is at least partially coated with or made from a substance that is superhydrophobic, superoleophobic, or both. In an embodiment, contact portion 29 of implantable medical device 15 is at least partially coated with or made from a plurality of substances that are superhydrophobic, superoleophobic, or both.

Additional embodiments of medical device 1 include an electrophysiology catheter; a self-expanding stent delivery system; a braided metal implant; a flow diverter (e.g., PIPELINE, from Covidien); a neurological stent (e.g., SILK from Balt); a multi electrode electrophysiology mapping and ablation device; a knitted polymer filament mesh device, e.g., for hernia repair; a urogyncologic sling, a prolapse device; a cosmetic surgery mesh; a device made of a noble metal; or the like.

In an embodiment, a portion of medical device 1 is at least partially coated with or made from a substance that is superhydrophobic, superoleophobic, or both. Suitable portions of a medical device include: a luminal surface of a coronary stent; a luminal surface of a percutaneous valve delivery catheter; a distal luminal surface where a preloaded implant is in contact with the delivery catheter; a luminal surface of an angiographic or infusion catheter; a fixation pin for a fixation device; an articulated surface of a joint implant; a lumen of a self-expanding stent delivery system; a surface or surface of a self-expanding stent delivery system; an abdominal aortic aneurysm delivery system; an AAA graft; a septal defect device; a mesh contacting an angiography catheter (e.g., HD MAPPER™ catheter from Bard); or the like.

In an embodiment the substance that is superhydrophobic, superoleophobic, or both provides a hemocompatible (blood compatible) surface to the medical device. For example, a medical device with a hemocompatible coating can reduce effects that may be associated with placing a foreign object in contact with blood components, such as the formation of thrombus or emboli (blood clots that release and travel downstream.

In certain embodiments, a superhydrophobic surface or coating, a superoleophobic surface or coating, a coating or surface that is both superhydrophobic and superoleophobic exhibits a static contact angle>150° as measured by water in air.

### Medical Devices

The present invention relates to any of a variety of medical devices that can include a coating or surface that is superhydrophobic, superoleophobic, or both. Suitable medical devices (e.g., embodiments of medical device 1) include implantable devices and non-implantable medical devices.

Embodiments of the invention can include and can be used with implantable, or transitorily implantable, devices including, but not limited to, vascular devices such as grafts (e.g., abdominal aortic aneurysm grafts, etc.), stents (e.g., self-expanding stents typically made from nitinol, balloon-expanded stents typically prepared from stainless steel, degradable coronary stents, etc.), catheters (including arterial, intravenous, blood pressure, stent graft, etc.), valves (e.g., polymeric or carbon mechanical valves, tissue valves, valve designs including percutaneous, sewing cuff, and the like), embolic protection filters (including distal protection devices), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies (e.g., batteries, etc.), peripheral cardiovascular devices, atrial septal defect closures, left atrial appendage filters, valve annuloplasty devices (e.g., annuloplasty rings), mitral valve repair devices, vascular intervention devices, ventricular assist pumps, and vascular access devices (including parenteral feeding catheters, vascular access ports, central venous access catheters); surgical devices such as sutures of all types, staples, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps; orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, implanted drug infusion tubes, drug infusion catheters, and intravitreal drug delivery devices; ophthalmic devices including orbital implants, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices; synthetic prostheses such as breast prostheses and artificial organs (e.g., pancreas, liver, lungs, heart, etc.); respiratory devices including lung catheters; neurological devices such as neurostimulators, neurological catheters, neurovascular balloon catheters, neuro-aneurysm treatment coils, and neuropatches; ear nose and throat devices such as nasal buttons, nasal and airway splints, nasal tampons, ear wicks, ear drainage tubes, tympanostomy vent tubes, otological strips, laryngectomy tubes, esophageal tubes, esophageal stents, laryngeal stents, salivary bypass tubes, and tracheostomy tubes; biosensor devices including glucose sensors, cardiac sensors, intra-arterial blood gas sensors; oncological implants; and pain management implants.

Classes of non-implantable devices can include dialysis devices and associated tubing, catheters, membranes, and grafts; autotransfusion devices; vascular and surgical devices including atherectomy catheters, angiographic catheters, intraaortic balloon pumps, intracardiac suction devices, blood pumps, blood oxygenator devices (including tubing and membranes), blood filters, blood temperature monitors, hemoperfusion units, plasmapheresis units, transition sheaths, dialators, intrauterine pressure devices, clot extraction catheters, percutaneous transluminal angioplasty catheters, electrophysiology catheters, breathing circuit connectors, stylets (vascular and non-vascular), coronary guide wires, peripheral guide wires; dialators (e.g., urinary, etc.); surgical instruments (e.g. scalpels and the like); endoscopic devices (such as endoscopic surgical tissue extractors, esophageal stethoscopes); and general medical and medically related devices including blood storage bags, umbilical tape, membranes, gloves, surgical drapes, wound dressings, wound management devices, needles, percutaneous closure devices, transducer protectors, pessary, uterine bleeding patches, PAP brushes, clamps (including bulldog clamps), cannulae, cell culture devices, materials for in vitro diagnostics, chromatographic support materials, infection control devices, colostomy bag attachment devices, birth control devices; disposable temperature probes; and pledgets.

In some aspects, embodiments of the invention can include and be utilized in conjunction with ophthalmic devices. Suitable ophthalmic devices in accordance with these aspects can provide bioactive agent to any desired area of the eye. In some aspects, the devices can be utilized to deliver bioactive agent to an anterior segment of the eye (in front of the lens), and/or a posterior segment of the eye (behind the lens). Suitable ophthalmic devices can also be utilized to provide bioactive agent to tissues in proximity to the eye, when desired.

In some aspects, embodiments of the invention can be utilized in conjunction with ophthalmic devices configured for placement at an external or internal site of the eye. Suitable external devices can be configured for topical administration of bioactive agent. Such external devices can reside on an external surface of the eye, such as the cornea (for example, contact lenses) or bulbar conjunctiva. In some embodiments, suitable external devices can reside in proximity to an external surface of the eye.

Devices configured for placement at an internal site of the eye can reside within any desired area of the eye. In some aspects, the ophthalmic devices can be configured for placement at an intraocular site, such as the vitreous. Illustrative intraocular devices include, but are not limited to, those described in U.S. Pat. Nos. 6,719,750 B2 (“Devices for Intraocular Drug Delivery,” Varner et al.) and 5,466,233 (“Tack for Intraocular Drug Delivery and Method for Inserting and Removing Same,” Weiner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and U.S. application Ser. Nos. 11/204,195 (filed Aug. 15, 2005, Anderson et al.), 11/204,271 (filed Aug. 15, 2005, Anderson et al.), 11/203,981 (filed Aug. 15, 2005, Anderson et al.), 11/203,879 (filed Aug. 15, 2005, Anderson et al.), 11/203,931 (filed Aug. 15, 2005, Anderson et al.); and related applications.

Suitable ophthalmic devices can be configured for placement within any desired tissues of the eye. For example, ophthalmic devices can be configured for placement at a subconjunctival area of the eye, such as devices positioned extrasclerally but under the conjunctiva, such as glaucoma drainage devices and the like.

The type of device upon which a coating is formed can be described in terms of its configuration or architecture. For example, some exemplary insertable or implantable medical devices have a complex geometry, or an inner surface. “Inner surfaces” of devices are those surfaces in which only a limited amount of light, or no light, can be provided using conventional irradiation equipment. In other words, while conventional irradiation equipment can provide an ample amount of light to an outer surface of a device to immobilize a photoactivatable reagent, the same amount of light is not able to be provided to an inner surface to affect bonding and provide a comparable coated surface. Particular examples of substrates that have inner surfaces may include, for example, stents, catheters such as PTCA catheters and hemodialysis catheters, hemodialysis membranes, and other devices having inner surfaces. These substrates can be formed, for example, from a complex architecture of materials, may contain many pores, or have a lumen.

A device formed of a fabric, or that has fabric-like qualities, can reflect the complex geometry. The implantable device can be formed from textiles, which include woven materials, knitted materials, and braided materials. Particularly useful textile materials are woven materials which can be formed using any suitable weave pattern known in the art. The porous structure can be that of a graft, sheath, cover, patch, sleeve, wrap, casing, and the like, including many of the medical articles described herein. These types of articles can function as the medical article itself or be used in conjunction with another part of a medical article.

The present medical device can be made or coated by any of a variety of methods. Such methods include those described in U.S. Patent Nos. U.S. Pat. No. 7,556,710 (Leeflang et al.; filed Jan. 26, 2006), 7,553,387 (Leeflang et al.; filed Jan. 26, 2006), and 7,550,053 (Leeflang et al.; filed Feb. 2, 2007) and U.S. Patent Application Publication No. 2009/0126862 (Leeflang; filed Oct. 20, 2008); the disclosures of which are incorporated herein by reference.

### Additional Embodiments of Materials or Surfaces That Are Superhydrophobic, Superoleophobic, or Both

Additional suitable coatings, materials, or surfaces that are superhydrophobic, superoleophobic, or both include so-called SLIPS materials. SLIPS materials are slippery liquid-infused porous surfaces. In certain embodiments, SLIPS materials can be one or more of pressure-stable, effectively repairable, foul-resistant, or transparent. These materials include a porous material and a lubricating fluid. Together the porous material and the lubricating fluid provide a coating, material, or surface that is superhydrophobic, superoleophobic, lubricious, or a combination thereof. Suitable porous materials include elctrospun mesh, such as those made from fluorinated polymers; filter paper, such as those provided by Whatman; other porous cellulosic materials; structured surfaces (e.g., as described below); porous metal oxide surfaces, such as those made from ZnO, TiO₂; polyvinyl difluoride (PVDF); and the like. Suitable lubricating fluids include a perfluorinated ionic liquid, such as, for example, 1-butyl-3-methylimidazolium hexafluorophosphate.

Structured surfaces can also provide coatings, materials, or surfaces that are superhydrophobic, superoleophobic, or both. Suitable structure surfaces include those described in L. Mischchenko et al. ACS Nano 4 (12), 7699-7707 (2010), the disclosure of which is incorporated herein by reference. Suitable silicon nanostructures can be fabricated according to the Bosch process (e.g., as described in Krupenkin, T. N.; Taylor, J. A.; Wang, E. N.; Kolodner, P.; Hodes, M.; Salamon, T. R. Reversible Wetting-Dewetting Transitions on Electrically Tunable Superhydrophobic Nanostructured Surfaces. Langmuir 2007, 23, 9128-9133). These nanostructures are then treated with a hydrophobic silane (e.g., tridecafluoro-1,1,2,2-tetrahydrooctyl)-trichlorosilane) by vapor exposure in a desiccator under vacuum overnight.

These structured surfaces can have geometrical features in the form of staggered bricks (e.g., subway brick pattern), posts, wide posts, blades, or honeycomb. Suitable geometrical features can be described by pitch, height, and wall/post thickness ratio of, for example (all dimensions are in μm):

| | | | Wall or Post | |
|---|---|---|---|---|
| Geometry | Pitch | Height | Thickness | φ-Ratio |
| staggered brick | 38.5 and 15.4 | 10.9 | 1.4 | 0.1 |
| post | 3.6 | 9.9 | 1.5 | 0.1 |
| wide post | 16.2 | 7.8 | 4.5 | 0.1 |
| blade | 5.2 | 6 | 1 | 0.2 |
| honeycomb | 34.5 | 7.5 | 3.3 | 0.4 |

Another suitable material is described in I. S. Bayer et al. Applied Physics Express 2 (2009) 125003, the disclosure of which is incorporated herein by reference. Such a material can include nanostructured superhydrophobic polymer-organo clay films including anaerobic acrylic adhesive, epoxy adhesive, urethane adhesive, cyano acrylate adhesive, and the like. Such materials can display strong adhesion to metal surfaces. Such adhesives can include those employed in bone cements. Montmorillonite clay filled anaerobic adhesives can be modified by blending with a water dispersed fluoromethacrylic latex in solution to form abrasion resistant interpenetrating polymer network films upon spray casting.

Organically modified nanostructured montmorillonite can be dispersed in anaerobic acrylic adhesives and subsequently blended with water borne fluoromethacrylic latex (e.g., Zonyl 8740) in alcohol solutions. The coatings can thermoset on aluminum surfaces under oxygen-rich conditions. No post-surface treatment is needed to render them superhydrophobic. Any of a variety of commercially available high-strength anaerobic adhesives can be employed, including those containing liquid polyester resins. Dimethyl dialkyl amine functionalized (35-45 wt %) montmorillonite clay particles can be dispersed in dimethyl sulfoxide (DMSO) at 0.25 g/ml. To this, can be added anaerobic bioadhesive (e.g., bone cement), which can be a blend of poly(ethylene glycol)dimethacrylate (PECDMA) and a polyester functional acrylic oligomer. A suitable composition includes PECDMA:CN7 10:CHP:polyamidc-wax:propylene glycol:fumed silica at 75:15:3:3:3:1 by weight percent. In an embodiment, the bone adhesive can include or be standard PMMA containing adhesive. The organoclay-bioadhesive dispersion in DMSO can be diluted with ethanol to a final nanoclay concentration of 0.1 g/ml and adhesive concentration of ˜5% by volume. The diluted organoclay-bioadhesive dispersion can be blended with waterborne fluoromethacrylic latex.

Another suitable material is described in I. S. Bayer et al. Applied Surface Science 257 (2010) 823-826; the disclosure of which is incorporated herein by reference. Nano-structured polyurethane/organoclay composite films can be fabricated by dispersing moisture curable polyurethanes and fatty amine/amino-silane surface modified montmorillonite clay (organoclay) in cyclomethicone-in-water emulsions. Cyclomethicone Pickering emulsions can be made by emulsifying decamethylcyclopentasiloxane (D5), dodecamethylcyclohexasiloxane (D6) and aminofunctional siloxane polymers with water using montmorillonite particles as emulsion stabilizers. Polyurethane and organoclay dispersed emulsions can be spray coated on aluminum surfaces. Upon thermosetting, water repellent self-cleaning coatings can be obtained. Moisture-curable polyurethane can be provided as a one-component liquid formula comprising 25% diphenylmethane-diisocyanate and 75% polyurethane pre-polymer (hexanedioic acid, polymer with 1,6-hexanediol and 1,1-methylenebis 4-isocyanatobenzene). The ingredients can be mixed until the emulsion is partially homogenous and then sonicated to stabilize it. The viscosity can be reduced to a desired level with ethyl acetate for spraying. The organoclay can be treated with benzyl alcohol before use. The ingredients and their weight percentages in the composition can be: Deionized water, 60; Decamethylcyclopentasiloxane (D5) oil, 12; Dodecamethylcyclohexasiloxane (D6) oil, 10; Petroleum distillates, 5; Naphta, 3; Montmorillonite clay, 3; Aminofunctional siloxanes, 3; Isopropyl alcohol, 4.

### Photoactivatable Groups

In certain embodiments, the coatings, materials, or surfaces that are superhydrophobic, superoleophobic, or both are derivatized with one or more photoactivatable group(s). Exemplary photoreactive groups that can be pendent from the coatings, materials, or surfaces that are superhydrophobic, superoleophobic, or both include those described in U.S. Pat. No. 5,414,075 and in U.S. patent application Ser. No. 13/490,994 (to Swan et al. and filed Jun. 7, 2012), the disclosures of which is incorporated herein by reference.

This material includes a chemical backbone having attached to it one or more first latent reactive groups and one or more second latent reactive groups, each of the first and second latent reactive groups being attached to the backbone in such a manner that, upon activation of the latent reactive groups in the presence of a support surface, a) the first latent reactive groups are capable of covalently bonding to the support surface, and b) upon bonding of the first latent reactive groups to the surface, the second latent reactive groups are; i) restricted from reacting with either a spacer or the support surface, ii) capable of reverting to their inactive state, and iii) upon reverting to their inactive state, are thereafter capable of being reactivated in order to later bind a target molecule, thereby attaching the target molecule to the surface.

In a particularly preferred embodiment, the chemical backbone of such a multifunctional reagent is a single tetrahedral carbon atom. Attached to the central carbon, in this embodiment, are four identical latent reactive groups, in the form of photoreactive groups, each attached via identical spacer chains. Upon exposure to a suitable light source, each of the latent reactive groups are subject to activation.

By virtue of conformational and/or steric constraints that the reagent imposes on itself (hence “restrained”), both by the tetrahedral nature of the central carbon, as well as the physical-chemical nature of the spacer chains themselves (e.g., their length, reactivity, and flexibility), the reagent is restricted, in that a maximum of three of the four activated latent reactive groups on any given preferred reagent molecule are able to attach to the support surface. The remaining unreacted group(s) are thus able to revert to their inactive state. In a subsequent step, the unreacted group(s) can be reactivated in the presence of a target molecule, in order to covalently bond the target molecule to the surface.

The reagent of the present invention involves a chemical backbone having attached to it one or more first latent reactive groups capable of attaching to a surface, and one or more second latent reactive groups capable of attaching to a target molecule intended for immobilization. Chemically, the first and second latent reactive groups, and respective spacers, can be the same or different.

In situations in which all latent reactive groups and spacers are chemically, or at least functionally, the same, the distinction between first and second latent reactive groups may actually be accomplished at the time of the first activation step, i.e., those groups that are activated and attach to the surface will be considered “first” latent reactive groups, and those that remain unreacted (whether or not they have been activated) will be considered “second” latent reactive groups.

The first and second latent reactive groups are preferably attached to the backbone by spacer chains in such a manner that, upon activation of the latent reactive groups in the presence of a support surface, the first latent reactive groups are capable of covalently bonding to the surface. The second latent reactive groups are thereby conformationally restricted, thus preventing reaction with either their spacers, other restricted reagents of the same type, or the support surface. In addition, after the first activation step and removal of the activating stimulus (e.g., illumination source), the second latent reactive groups are capable of reverting to their inactive state and can thereafter be activated (or reactivated, as the case may be) to covalently bond a target molecule.

The following diagram depicts the concept of the preferred tetrahedral core structure, as exemplified by the empirical formula X(Y)₄(Z)₄, shown below as Formula I:

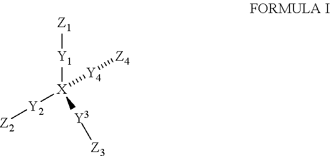

In Formula I:

X=the chemical backbone;

Y₁, Y₂, Y₃, Y₄=optional spacers; and

Z₁, Z₂, Z₃, Z₄=latent reactive groups.

In an embodiment, the invention provides a core molecule containing four dimethyleneoxy groups bonded as spacers to a central tetrahedral carbon atom, the carbon atom serving in this instance as the chemical backbone. The backbone, spacers, and latent reactive groups are described herein, for the sake of simplicity, as being distinct portions of the reagent of the present invention. In the chemical synthesis of a reagent however, these portions will rarely be provided as three independent precursors. Instead, and most often, the portion referred to herein as the spacer will be formed as the result of the reaction between two molecules, one that contains the core molecule and another that contains the latent reactive group.

By virtue of the physical and chemical properties of the photoreactive groups and the methylene group spacers, together with the conformational restrictions provided by the tetrahedral carbon backbone, the reagent is able to attach up to three of its photoreactive groups to a surface upon photoactivation. Being conformationally restricted, and thus unable to interact with the support surface or the spacers, any remaining photoreactive group(s) are able to return to their inactive states upon removal of fight, once again being capable of activation by subsequent illumination.

In addition to reagents of the particularly preferred embodiment, containing a central carbon atom, reagents of the present invention can be prepared having any suitable chemical (e.g., organic and/or inorganic) backbone structure, including those that employ a single atom, such as silicon, nitrogen, phosphorus, and any other atom with four or more bonds nonplanar with respect to one another.

Also, molecules having conformationally restricted ring structures (such as inositol, i.e., hexahydroxy cyclohexane) can be derivatized with latent reactive groups in a manner analogous to that described herein for pentaerythritol, to provide latent reactive groups in both axial and equatorial positions. Other polyhydroxylated compounds such as mono- and di-saccharides, and cyclodextrins, are suitable as well, in that they offer alternative opportunities to create other multisubstituted reagents having varying placements and densities of latent reactive groups.

Contact with a support surface and activation of the latent reactive groups will result in covalent bond formation through at least one latent reactive group, with at least one other latent reactive group being conformationally restricted and thus unable to react at the surface.

Spacers useful in the reagent of the present invention can be bonded to the tetrahedral atom and can be of any suitable length and structure. A “spacer”, as used herein, refers to that region of a reagent between a latent reactive group and a chemical backbone. The use of spacers is optional, and would not be necessary, for instance, for such compounds as acylated derivatives of tetraphenylmethane having the structure shown below as Formula II:

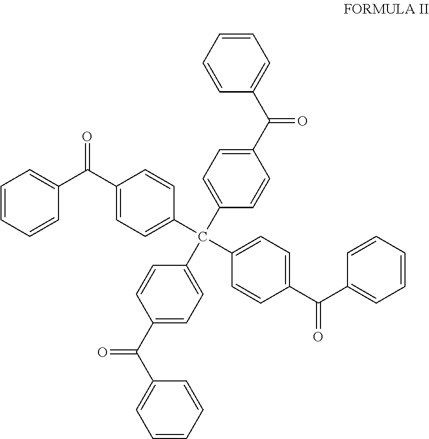 [SMILES: O=C(c1ccccc1)c1ccc(C(c2ccc(C(=O)c3ccccc3)cc2)(c2ccc(C(=O)c3ccccc3)cc2)c2ccc(C(=O)c3ccccc3)cc2)cc1]

A “latent reactive group”, as used herein, refers to a chemical group that responds to an applied external energy source in order to undergo active specie generation, resulting in covalent bonding to an adjacent chemical structure (e.g., an abstractable hydrogen). Preferred groups are sufficiently stable to be stored under conditions in which they retain such properties. See, e.g., U.S. Pat. No. 5,002,582, the disclosure of which is incorporated herein by reference. Latent reactive groups can be chosen that are responsive to various portions of the electromagnetic spectrum, with those responsive to ultraviolet and visible portions of the spectrum (referred to herein as “photoreactive”) being particularly preferred.

Photoreactive aryl ketones such as acetophenone and benzophenone, or their derivatives, are preferred, since these functional groups, typically, are readily capable of undergoing the activation/inactivation/reactivation cycle described herein. Benzophenone is a particularly preferred photoreactive group, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (e.g., carbon-hydrogen) is not available for bonding, the ultraviolet light-induced excitation of the benzophenone group is reversible and the molecule returns to ground state energy level upon removal of the energy source. Hence, photoreactive aryl ketones are suitable.

A linking agent suitable for use in the present material is described in U.S. Pat. No. 5,714,360, the disclosure of which is incorporated herein by reference.

A chemical linking agent including a di- or higher functional photoactivatable charged compound can be employed. This linking agent provides at least one group that is charged under the conditions of use in order to provide improved water solubility. The agent further provides two or more photoactivatable groups in order to allow the agent to be used as a cross-linking agent in aqueous systems. In an embodiment, the charge is provided by the inclusion of one or more quaternary ammonium radicals, and the photoreactive groups are provided by two or more radicals of an aryl ketone such as benzophenone.

In a preferred embodiment, the invention provides a linking agent of the general formula: X—Y—X; wherein each X, independently, is a radical containing a photoreactive group and Y is a radical containing, inter alia, one or more charged groups. In such an embodiment, the number and/or type of charged group(s) is sufficient to provide the molecule with sufficient aqueous solubility to allow the agent to be used (i.e., applied to a surface and activated) in a solvent system having water as a major component.

In an embodiment, Y contains one or more nitrogen-containing (e.g., quaternary ammonium) groups. For example, Y contains a linear or heterocyclic radical selected from the group consisting of:

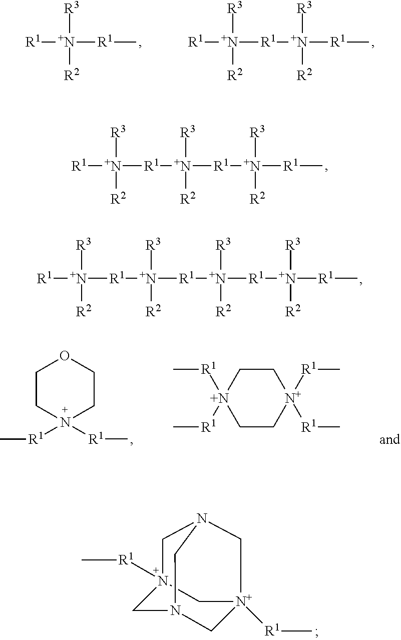

wherein each R¹ independently is a radical containing an alkylene, oxyalkylene, cycloalkylene, arylene, or aralkylene group, each R² independently is a radical containing an alkyl, oxyalkyl, cycloalkyl, aryl, or aralkyl group, and each R³ independently is either a non-bonding pair of electrons, a hydrogen atom, or a radical of the same definition as R², in which the R¹, R² and R³ groups can contain noninterfering heteroatoms such as O, N, S, P and the like, and/or noninterfering substituents such as halo (e.g., Cl) and the like.

In an embodiment, one or more R² radicals contains an aralkyl group in the form of a photoactivatable aryl ketone. These groups, in addition to the two photoactivatable groups provided by the above-defined X groups, can be used to provide the “triphoto”, “tetraphoto” and higher order photoactivatable groups described herein. The use of three or more total photoreactive groups provides the linking agent with further ability to cross-link the agent to a target molecule and/or to a surface.

In yet another preferred embodiment, the R² and R³ groups of the above linear radicals can, in effect, be fused (e.g., an R² and an R³ on a single N atom, or a suitable combination of R²/R³ groups on adjacent N atoms) in order to form heterocyclic structures other than those exemplified above. The specific choice and relationship between R groups in a linking agent of the present invention is not critical, so long as the linking agent provides two or more photoactivatable groups and retains sufficient water solubility for its intended use.

### Linking Agent

A water-soluble, linking agent suitable for use as the present device is described in U.S. patent application Ser. No. 13/074,537 (Kurdymov et al.; filed Mar. 29, 2011), the disclosure of which is incorporated herein by reference.

The linking agent can have the formula Photo¹-LG-Photo², wherein Photo¹ and Photo², independently, represent at least one photoreactive group and LG represents a linking group. In one embodiment, one or more photoreactive groups include an aryl ketone. In a more particular embodiment, one or more photoreactive groups include benzophenone.

In one embodiment, the linking group includes one or more silicon atoms or one or more phosphorus atoms, wherein each photoreactive group is independently bonded to the linking group by a covalent linkage that includes at least one heteroatom. In one embodiment, at least one heteroatom is selected from oxygen, nitrogen, selenium, sulfur, or a combination thereof. In one embodiment, at least one photoreactive group, heteroatom and linking group form an ether or an amine.

In a more particular embodiment, the linking group includes one silicon atom covalently bonded to at least two photoreactive groups. In another embodiment, the linking group includes at least two silicon atoms. In another embodiment, the linking group has the formula Si—Y—Si, wherein Y represents a linker that can be null, an amine, ether, linear or branched C₁-C₁₀ alkyl, or a combination thereof. In one embodiment, Y is selected from O, CH₂, OCH₂CH₂O and O(CH₂CH₂O)ₙ, wherein n is an integer between 1 and 5, between 1 and 10, between 1 and 15, between 1 and 20, between 1 and 25, or between 1 and 30.

In another embodiment, the linking group includes one or more phosphorester bonds and/or one or more phosphoramide bonds wherein one or more phosphorester and/or one or more phosphoramide bonds form a covalent bond with at least one photoreactive group, such that the linking group includes at least two photoreactive groups. In one embodiment, the linking group is covalently attached to three photoreactive groups, wherein each photoreactive group is covalently bonded to the linking group by a phosphorester or phosphoramide bond. In another embodiment, the linking group includes at least one phosphorus atom with a phosphorus-oxygen double bond (P═O), wherein at least one photoreactive group is bonded to at least one phosphorus atom. In yet another embodiment, the linking group includes one phosphorus atom with a phosphorus-oxygen double bond (P═O), wherein at least two or three photoreactive groups are covalently bonded to the phosphorus atom. In another embodiment, the linking group includes at least two phosphorus atoms, wherein at least one phosphorus atom includes a phosphorus-oxygen double bond (P═O), and at least one or at least two photoreactive groups are covalently bonded to each phosphorus atom.

The linking agent includes one or more photoreactive groups and a linking group, wherein each photoreactive group is independently attached to the linking group by a linkage. In other embodiments, the linking agent includes two or more photoreactive groups. In still other embodiments, the linking agent includes three or more photoreactive groups.

The linking agent includes one or more photoreactive groups attached to a linking group. The linking agent can be represented by the formula Photo¹-LG-Photo², wherein Photo¹ and Photo² independently represent at least one photoreactive group and LG represents a linking group. The term “linking group” as used herein, refers to a segment or group of molecules configured to connect two or more molecule to each another, wherein the linking group is capable of degrading under one or more conditions. In one embodiment, the linking group includes at least one silicon atom. In another embodiment, the linking group includes at least one phosphorus atom.

The term “linking group” as used herein, refers to a moiety configured to connect one molecule to another, wherein the linking group is capable of cleavage under one or more conditions. The term “biodegradable” as used herein, refers to degradation in a biological system, and includes for example, enzymatic degradation or hydrolysis. It should be noted that the term “degradable” as used herein includes both enzymatic and non-enzymatic (or chemical) degradation. It is also understood that hydrolysis can occur in the presence of or without an acid or base. In one embodiment, the linking agent is water soluble. In another embodiment, the linking agent is not water soluble.

In addition to providing a bond, the linking group can function as a spacer, for example, to increase the distance between the photoreactive groups of the linking agent. For example, in some instances it may be desirable to provide a spacer to reduce steric hindrance that may result between the photoreactive groups, which could interfere with the ability of the photoreactive groups to form covalent bonds with a support surface, or from serving as a photoinitiator for polymerization. As described herein, it is possible to vary the distance between the photoreactive groups, for example, by increasing or decreasing the spacing between one or more photoreactive groups.

As described herein, one or more photoreactive groups can be bonded to a linking group by a linkage. In one embodiment, the linkage between the photoreactive group and the linking group includes at least one heteroatom, including, but not limited to oxygen, nitrogen, selenium, sulfur or a combination thereof. In one embodiment, a photoreactive group, linking group and heteroatom form an ether (R¹—O—R²), wherein R¹ is a photoreactive group and R² is a linking group. In another embodiment, a photoreactive group, linking group and heteroatom form an amine,

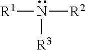

wherein R¹ is a photoreactive group, R² is a linking group, and R³ is hydrogen, aryl or alkyl, a photoreactive group, or a hydroxyl or salt thereof. In one embodiment, R³ is cyclic, linear or branched, saturated or unsaturated, aromatic or heteroaromatic, or a combination thereof. The stability of the ether and/or amine linkage can be influenced depending upon the size (e.g., chain length, branching, bulk, etc.) of the substituents. For example, bulkier substituents will generally result in a more stable linkage (i.e., a linking agent that is slower to degrade in the presence of water and/or acid).

In one embodiment, the linking group includes one or more silicon atoms. In a particular embodiment, the linking group includes one silicon atom (which can be referred to as a monosilane) covalently bonded to at least two photoreactive groups. In another embodiment, the linking group includes at least two silicon atoms (which can be referred to as a disilane). In one embodiment, the linking group can be represented by the formula Si—Y—Si, wherein Y represents a linker that can be null (e.g., the linking group includes a direct Si—Si bond), an amine, ether, linear or branched C₁-C₁₀ alkyl, or a combination thereof. In one embodiment, Y is selected from O, CH₂, OCH₂CH₂O, O(CH(CH3)CH₂O)ₙ, and O(CH₂CH₂O)ₙ, wherein n is an integer between 1 and 5, between 1 and 10, between 1 and 15, between 1 and 20, between 1 and 25, or between 1 and 30. One embodiment of a disilane linking agent is shown below

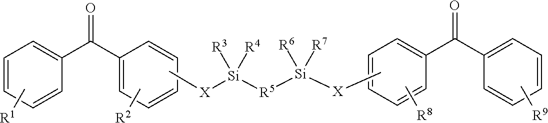

wherein R¹, R², R⁸ and R⁹ can be any substitution, including, but not limited to H, alkyl, halide, hydroxyl, amine, or a combination thereof; R³, R⁴, R⁶ and R⁷ can be alkyl, aryl or a combination thereof; R⁵ can be any substitution, including but not limited to O, alkyl or a combination thereof and each X, independently, can be O, N, Se, S, or alkyl, or a combination thereof. One specific embodiment is shown below:

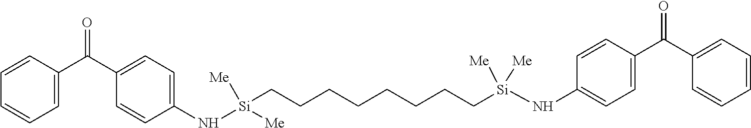 [SMILES: C[Si](C)(CCCCCCCC[Si](C)(C)Nc1ccc(C(=O)c2ccccc2)cc1)Nc1ccc(C(=O)c2ccccc2)cc1]

In one embodiment, the linking agent can be represented by the formula

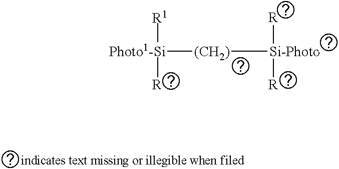

wherein Photo¹ and Photo², independently, represent one or more photoreactive groups and n is an integer between 1 and 10, wherein the linking agent comprises a covalent linkage between at least one photoreactive group and the linking group, wherein the covalent linkage between at least one photoreactive group and the linking group is interrupted by at least one heteroatom. In general, a longer hydrocarbon chain between the two silicon atoms will tend to increase the flexibility of the linking agent and may facilitate crosslinking between a greater number of polymers than a linking agent with a shorter carbon chain, since the photoreactive groups can react with polymers located farther apart from one another. In the formula shown above, R¹, R², R³, R⁴ are independently alkyl or aryl, including, but not limited to cyclic, linear or branched, saturated or unsaturated, aromatic or heteroaromatic, or a combination thereof. In a more particular embodiment, R¹-R⁴ are independently phenyl, methyl, ethyl, isopropyl, t-butyl, or a combination thereof. In another embodiment, R¹-R⁴ can also be, independently, a photoreactive group. In yet another embodiment, R¹-R⁴ can also be, independently, hydroxyl or salt thereof. In one embodiment, the hydroxyl salt includes a counterion that is lithium, sodium, potassium, or a combination thereof.

In another embodiment, the linking agent can be represented by the formula

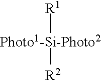

wherein Photo¹ and Photo², independently, represent one or more photoreactive group, wherein the linking agent comprises a covalent linkage between at least one photoreactive group and the linking group, wherein the covalent linkage between at least one photoreactive group and the linking group is interrupted by at least one heteroatom; R¹ and R² are independently alkyl or aryl, including, but not limited to cyclic, linear or branched, saturated or unsaturated, aromatic or heteroaromatic, or a combination thereof. In a more particular embodiment, R¹ and R² are independently phenyl, methyl, ethyl, isopropyl, t-butyl, or a combination thereof. R¹ and R² can also be, independently, a photoreactive group, wherein the linking agent comprises a covalent linkage between at least one photoreactive group and the linking group, wherein the covalent linkage between at least one photoreactive group and the linking group is interrupted by at least one heteroatom; or hydroxyl or salt thereof. In one embodiment, the hydroxyl salt includes a counterion that is lithium, sodium, potassium, or a combination thereof. One embodiment of a monosilane linking agent is shown below

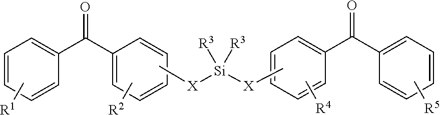

in which R¹ and R⁵ can be any substitution, including, but not limited to H, halogen, amine, hydroxyl, alkyl, or a combination thereof; R² and R⁴ can be any substitution, except OH, including, but not limited to H, alkyl or a combination thereof; R³ can be alkyl, aryl or a combination thereof; and X, independently, can be O, N, Se, S, alkyl or a combination thereof.

In another embodiment, the linking group includes one or more phosphorous atoms. In one embodiment, the linking group includes one phosphorus atom (which can also be referred to as a mono-phosphorus linking group). In another embodiment, the linking agent includes two phosphorus atoms (which can also be referred to as a bis-phosphorus linking group). In one embodiment, the linking group comprises at least one phosphorus atom with a phosphorus-oxygen double bond (P═O), wherein at least one or two photoreactive groups are bonded to the phosphorus atom. In another embodiment, the linking group comprises one phosphorus atom with a phosphorus-oxygen double bond (P═O), wherein two or three photoreactive groups are covalently bonded to the phosphorus atom. In another embodiment, the linking group comprises at least two phosphorus atoms, wherein at least one phosphorus atom includes a phosphorus-oxygen double bond (P═O), and at least one or two photoreactive groups are covalently bonded to each phosphorus atom.

In a more particular embodiment, the linking agent can be represented by the formula:

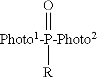

wherein Photo¹ and Photo², independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group, hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In a more particular embodiment, R is phenyl, methyl, ethyl, isopropyl, t-butyl, or a combination thereof.

In another embodiment, the linking agent can be represented by formula:

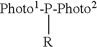

wherein Photo¹ and Photo² independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group (wherein the covalent linkage between the photoreactive group and the linking group may be interrupted by at least one heteroatom), hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In one embodiment, R is phenyl, methyl, ethyl, isopropyl, t-butyl, or a combination thereof.

In another embodiment, the linking agent can be represented by the formula:

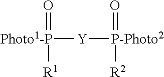

wherein Photo¹ and Photo², independently, represent one or more photoreactive groups, wherein the linking agent comprises a covalent linkage between at least one photoreactive group and the linking group, wherein the covalent linkage between at least one photoreactive group and the linking group is interrupted by at least one heteroatom; Y represents a linker that can be N or O (e.g., pyrophosphate), linear or branched C₁-C₁₀ alkyl, or a combination thereof; and R¹ and R² are independently alkyl, aryl, a photoreactive group (wherein the covalent linkage between the photoreactive group and the linking group can be interrupted by at least one heteroatom), hydroxyl or salt thereof, or a combination thereof. In one embodiment, Y is selected from O, CH₂, OCH₂CH₂O, O(CH(CH3)CH₂O)ₙ, and O(CH₂CH₂O)ₙ, wherein n is an integer between 1 and 5, between 1 and 10, between 1 and 15, between 1 and 20, between 1 and 25, or between 1 and 30. In one embodiment, the hydroxyl salt counterion is lithium, sodium, potassium, or a combination thereof. In a more particular embodiment, R¹ and R² are independently, cyclic, linear or branched hydrocarbon, saturated or unsaturated, aromatic or heteroaromatic, or a combination thereof. In one embodiment, R¹ and R² are independently phenyl, methyl, ethyl, isopropyl, t-butyl, or a combination thereof. In general, a longer hydrocarbon chain between the two phosphorus atoms will tend to increase the flexibility of the linking agent and may facilitate crosslinking between a greater number of polymers than a linking agent with a shorter carbon chain, since the reactive photoreactive groups can react with polymers located farther apart from one another. In one embodiment, Y can be O, CH₂, OCH₂CH₂O, O(CH₂(CH3)CH₂O)ₙ, and O(CH₂CH₂O)ₙ wherein n is an integer between 1 and 5, between 1 and 10, between 1 and 15, between 1 and 20, between 1 and 25, or between 1 and 30. One embodiment is shown below

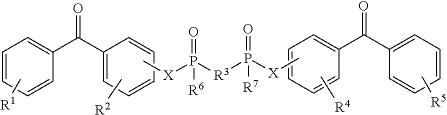

in which R¹, R², R⁴ and R⁵ can be any substitution, including but not limited to H, alkyl, halogen, amine, hydroxyl, or a combination thereof; R³ can be any substitution, including but not limited to O, alkyl, or a combination thereof; R⁶ and R⁷ can be alkyl, aryl or a combination thereof; and each X can independently be O, N, Se, S, alkyl, or a combination thereof. In one embodiment, the linking agent includes one or more phosphorester bonds and one or more phosphoramide bonds, and can be represented by the formula:

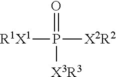

wherein X and X² are, independently, O, N, Se, S or alkyl; R¹ and R² are independently, one or more photoreactive groups, and X³ is O, N, Se, S, alkyl or aryl; R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or a hydroxyl or salt thereof. In one embodiment, the hydroxyl salt counterion is lithium, sodium, potassium, or a combination thereof.

In one embodiment, the linking agent comprises a triphosphorester, which can be represented by the formula.

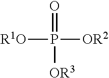

wherein R¹ and R² are independently, one or more photoreactive groups, and R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or a hydroxyl or salt thereof. In one embodiment, the hydroxyl salt counterion is lithium, sodium, potassium, or a combination thereof.

In another embodiment, the linking agent comprises a triphosphoramide, which can be represented by the formula.

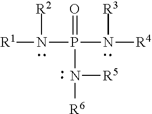

wherein R¹-R⁶ are independently, a photoreactive group, a hydroxyl or salt thereof, alkyl or aryl, or a combination thereof, wherein at least two of R¹-R⁶ are, independently, a photoreactive group. In one embodiment, the hydroxyl salt counterion is lithium, sodium, potassium, or a combination thereof. In a more particular embodiment, R¹-R⁶ are independently cyclic, linear or branched, saturated or unsaturated, aromatic or heteroaromatic, or a combination thereof. In a more particular embodiment, R¹-R⁶ are, independently, phenyl, methyl, ethyl, isopropyl, t-butyl, or a combination thereof.

The linking agent can be formed using any suitable reaction pathway. In one embodiment, the linking agent is formed by reacting a functionalized linking element with one or more, typically two or more photoreactive groups. As used herein, the term “linking element” refers to the linking group component of the linking agent before it is bonded to one or more photoreactive groups. The term “functionalized linking element” is used to indicate that the linking element includes one or more reactive functional groups. In one embodiment, the linking element includes one or more halogen functional groups. The term “halogen” refers to fluorine, chlorine, bromine, or iodine functional groups. In another embodiment, the linking element includes one or more trifluoromethanesulfonate (CF₃SO₃—) functional groups.

In one embodiment, the linking element includes one or more silicon atoms. In one embodiment, the linking element includes one or more halogen substituents, such as fluorine, chlorine, bromine, iodine, and combinations thereof. In another embodiment, the linking element includes at least two halogen substituents. In another embodiment, the linking element includes one or more trifluoromethanesulfonate (triflate) substituents. In another embodiment, the linking element includes at least two triflate substituents. In a more particular embodiment, the linking element includes one silicon atom with at least two halogen or triflate substituents. In another embodiment, the linking element includes at least two silicon atoms. In a more particular embodiment, the linking element includes two silicon atoms, wherein each silicon atom includes at least one halogen or triflate substituent. In one embodiment, the linking element can be represented by the formula Si—Y—Si, wherein Y represents a linker that can be null, an amine, ether, linear or branched C₁-C₁₀ alkyl, or a combination thereof, wherein each silicon atom includes at least one halogen or triflate substituent. In one embodiment, Y is selected from O, CH₂, OCH₂CH₂O, O(CH(CH3)CH₂O)ₙ, and O(CH₂CH₂O)ₙ, wherein n is an integer between 1 and 5, between 1 and 10, between 1 and 15, between 1 and 20, between 1 and 25, or between 1 and 30.

In one embodiment, the linking element can be represented by the formula

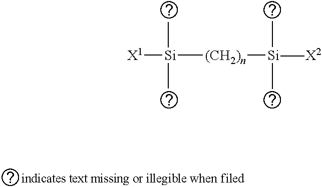

wherein X¹ and X² are independently halogen, such as fluorine, chlorine, bromine, iodine; trifluoromethanesulfonate; or a combination thereof and n is an integer between 1 and 10. R₁-R₄ are independently alkyl or aryl, including, but not limited to cyclic, linear or branched, saturated or unsaturated, aromatic or heteroaromatic, or a combination thereof. In a more particular embodiment, R¹-R⁴ are independently phenyl, methyl, ethyl, isopropyl, t-butyl, or a combination thereof. In another embodiment, R¹-R⁴ can also be, independently, halogen. In yet another embodiment, R¹-R⁴ can also be, independently, hydroxyl or salt thereof. In one embodiment, the hydroxyl salt includes a counterion that is lithium, sodium, potassium, or a combination thereof.

In another embodiment, the linking element can be represented by the formula

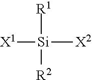

wherein X¹ and X² are independently halogen; such as fluorine, chlorine, bromine, and iodine; or trifluoromethanesulfonate; R¹ and R² are independently alkyl or aryl, including, but not limited to cyclic, linear or branched, saturated or unsaturated, aromatic or heteroaromatic, or a combination thereof. In a more particular embodiment, R¹ and R² are independently phenyl, methyl, ethyl, isopropyl, t-butyl, or a combination thereof. R¹ and R² can also be, independently, halogen, hydroxyl or hydroxyl salt. In one embodiment, the hydroxyl salt includes lithium, sodium, potassium, or a combination thereof as a counterion.

In another embodiment, the linking element includes one or more phosphorous atoms. In one embodiment, the linking element comprises at least one phosphorus atom with a phosphorus-oxygen double bond (P═O), wherein at least one halogen or trifluoromethanesulfonate substituent is bonded to at least one phosphorus atom. In another embodiment, the linking element comprises one phosphorus atom with a phosphorus-oxygen double bond (P═O), wherein two or three halogen or trifluoromethanesulfonate substituents are, independently, covalently bonded to the phosphorus atom. In another embodiment, the linking element comprises at least two phosphorus atoms, wherein at least one phosphorus atom includes a phosphorus-oxygen double bond (P═O), and at least one or two halogen or trifluoromethanesulfonate substituents are covalently bonded to each phosphorus atom. In a more particular embodiment, the linking element comprises two phosphorus atoms.

In a more particular embodiment, the linking element can be represented by the formula

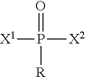

wherein X¹ and X² are independently halogen; such as fluorine, chlorine, bromine, and iodine; or trifluoromethanesulfonate; and R is alkyl or aryl, halogen, hydroxyl or a hydroxyl salt, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In a more particular embodiment, R is phenyl, methyl, ethyl, isopropyl, t-butyl, or a combination thereof.

In another embodiment, the linking element can be represented by formula:

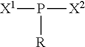

wherein X¹ and X² are independently halogen, such as fluorine, chlorine, bromine, and iodine; or trifluoromethanesulfonate and R is alkyl or aryl, halogen, trifluoromethanesulfonate, hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In one embodiment, R¹ and R² are independently phenyl, methyl, ethyl, isopropyl, t-butyl, or a combination thereof.

In another embodiment, the linking element can be represented by the formula:

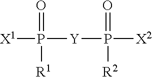

wherein X¹ and X² are independently halogen, such as fluorine, chlorine, bromine, and iodine; or trifluoromethanesulfonate, Y represents a linker that can be null, an amine, an ether, linear or branched C₁-C₁₀ alkyl, or a combination thereof; and R¹ and R² are independently alkyl, aryl, halogen, hydroxyl or salt thereof, or a combination thereof. In one embodiment, Y is selected from O, CH₂, OCH₂CH₂O, O(CH(CH3)CH₂O)ₙ, and O(CH₂CH₂O)ₙ, wherein n is an integer between 1 and 5, between 1 and 10, between 1 and 15, between 1 and 20, between 1 and 25, or between 1 and 30. In one embodiment, the hydroxyl salt counterion is lithium, sodium, potassium, or a combination thereof. In a more particular embodiment, R¹ and R² are independently, cyclic, linear or branched hydrocarbon, saturated or unsaturated, aromatic or heteroaromatic, or a combination thereof. In one embodiment, R¹ and R² are independently phenyl, methyl, ethyl, isopropyl, t-butyl, or a combination thereof.

### Water-Soluble, Degradable Linking Agent

A water-soluble, degradable linking agent suitable for use in the present polymeric medical device is described in U.S. Patent Application Nos. 61/285,345 and 61/358,464, the disclosure of which is incorporated herein by reference.

Described in this section is a linking agent that includes a core molecule with one or more charged groups; and one or more photoreactive groups covalently attached to the core molecule by one or more degradable linkers. In one embodiment, the linking agent includes a non-polymeric core molecule. In one embodiment, the non-polymeric core molecule is a hydrocarbon, including a hydrocarbon that is linear, branched, cyclic, or a combination thereof; aromatic, non-aromatic, or a combination thereof; monocyclic, polycyclic, carbocyclic, heterocyclic, or a combination thereof; benzene or a derivative thereof. In one embodiment, one or more degradable linkers comprise an amide, an ester, a thiocarbamate, or a combination thereof. In one embodiment, one or more photoreactive group is an aryl ketone, including, for example, acetophenone, benzophenone, anthraquinone, anthrone, anthrone-like heterocycles, substituted derivatives thereof, or a combination thereof. In one embodiment, one or more charged groups are negatively charged, including, for example, an organic acid selected from sulfuric acid, sulfonic acid, carboxylic acid, phosphoric acid, phosphonic acid, or a combination thereof. In another embodiment, one or more charged groups are positively charged, for example, a quaternary ammonium salt.

Described herein is a water-soluble, degradable linking agent. The degradable linking agent includes one or more photoreactive groups, one or more charged groups, and one or more degradable linkers configured to operably attach one or more photoreactive groups to one or more negatively charged groups. In one embodiment, the linking agent includes a core having one or more charged groups attached directly or indirectly thereto and one or more photoreactive groups attached to the non-polymeric core by one or more degradable linkers.

The degradable linking agent includes one or more photoreactive groups attached to one or more charged groups by a degradable linker. In a more particular embodiment, the degradable linking agent includes a core molecule to which the charged groups and the photoreactive groups can be independently attached. In one embodiment, the degradable linking agent includes a non-polymeric core molecule. The term “degradable linker” as used herein, refers to a segment configured to connect one part of the linking agent to another, wherein the linker is capable of cleavage under one or more conditions. The term degradable as used herein also encompasses “biodegradable linkers.” The term “biodegradable” as used herein, refers to degradation in a biological system, and includes for example, enzymatic degradation or hydrolysis. It should be noted that the term “degradable” as used herein includes both enzymatic and non-enzymatic (or chemical) degradation. In one embodiment, the degradable linker comprises one or more degradable linkages such as an amide, an ester, a thiocarbamate, or combinations thereof.

In addition to providing a degradable segment, the degradable linker can function as a spacer, to increase the distance between one or more photoreactive groups and the core molecule. For example, in some instances it may be desirable to provide a spacer to reduce steric hindrance that may result between the core molecule and one or more photoreactive groups that could interfere with the ability of one or more photoreactive groups to form covalent bonds with a support surface, or from serving as a photoinitiator for polymerization. As described herein, it is possible to vary the distance between the photoreactive groups, for example, by increasing or decreasing the spacing between one or more photoreactive groups.

A degradable linking agent can be represented by the formula:

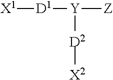

wherein X¹ and X² include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof D¹ and D² are, independently, degradable segments, including, for example, degradable segments that include an amide, an ester, a thiocarbamate, or a combination thereof; Y represents a core molecule, which can be either polymeric or non-polymeric, including, but not limited to a hydrocarbon, including a hydrocarbon that is linear, branched, cyclic, or a combination thereof aromatic, non-aromatic, or a combination thereof monocyclic, polycyclic, carbocyclic, heterocyclic, or a combination thereof benzene or a derivative thereof or a combination thereof and Z represents one or more charged groups, including, for example, one or more negatively charged groups such as an organic acid salt, including but not limited to sulfuric acid, sulfonic acid, carboxylic acid, phosphoric acid, phosphonic acid, or a combination thereof one or more positively charged groups, for example, a quaternary ammonium salt, or a combination thereof.

In the formula shown above, the two or more photoreactive groups (X¹ and X²) are discrete. As used herein, the term “discrete” means that the two or more photoreactive groups are distinct from each other, as compared to a bifunctional photoreactive agent, that can include two or more photoreactive moieties, such as a conjugated cyclic diketone wherein each ketone group of the diketone is adapted to serve as a photoreactive moiety capable of being activated in order to provide a free radical. It is also understood that the first and second photoreactive groups and/or the first and second degradable linkers may or may not be the same. For example, in one embodiment, the photoreactive groups (X¹ and X²) are the same or identical. In another embodiment, the photoreactive groups (X¹ and X²) are not the same. In one embodiment, the degradable linker (D¹ and D²) are the same or identical. In another embodiment, the degradable linker (D¹ and D²) are not the same. In one embodiment, the photoreactive groups include one or more first photoreactive groups adapted to attach the linking agent to a surface and one or more second photoreactive groups adapted to initiate photopolymerization.

In one embodiment, the degradable linker is a biodegradable linker that includes an amide bond (also referred to as a peptide bond, or peptide linker). A peptide bond can be cleaved by amide hydrolysis (the addition of water) by enzymatic and non-enzymatic reactions. Proteolysis refers to amide hydrolysis catalyzed by an enzyme. The term “protease” refers to an enzyme that conducts proteolysis. Examples of enzymes capable of hydrolyzing a peptide bond include, but are not limited to, acylase, amidohydrolase, deaminase, trypsin, and alpha-chymotrypsin.

A nonlimiting example of a degradable linker with a peptide bond can be represented by formula I:

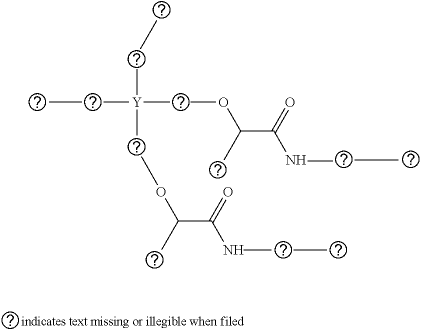

wherein X¹ and X² include, independently, one or more photoreactive groups, including, but not limited to, aryl ketone photoreactive groups, such as acetophenone, benzophenone, anthraquinone, anthrone, anthrone-like heterocycles, their substituted derivatives or a combination thereof; Y represents a core molecule, which can be polymeric or non-polymeric, including for example, non-polymeric molecules such as a hydrocarbon, including linear, branched or cyclic; aromatic or non-aromatic; monocyclic, polycyclic, carbocyclic or heterocyclic; benzene or a derivative thereof; or combinations thereof; Z¹ and Z² represent, independently, one or more charged groups, including positively and negatively charged groups, for example a negatively charged group that includes an organic acid salt, including but not limited to sulfuric acid, sulfonic acid, carboxylic acid, phosphoric acid, phosphonic acid, or a combination thereof; one or more positively charged groups, for example, a quaternary ammonium salt; or a combination thereof. R¹, R², R³, and R⁴ are, independently, spacer elements that can be null, a heteroatom, alkyl or aryl, including, but not limited to cyclic, linear or branched, saturated or unsaturated, aromatic or heteroaromatic, or a combination thereof; R⁵ and R⁶ are, independently, spacer elements that can be null, alkyl or aryl, including, but not limited to cyclic, linear or branched, saturated or unsaturated, aromatic or heteroaromatic, or a combination thereof; and R⁷ and R⁸ are, independently substituents that can be hydrogen, alkyl or aryl, including, but not limited to cyclic, linear or branched, saturated or unsaturated, aromatic or heteroaromatic, or a combination thereof.

More specific examples of a degradable linker that includes a degradable amide bond include those shown in formulae II and III:

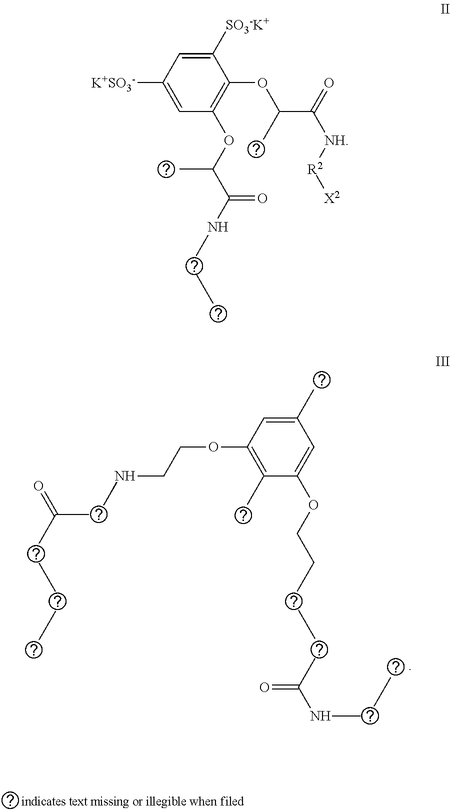

wherein X¹ and X² include, independently, one or more photoreactive groups, including, but not limited to aryl ketone photoreactive groups, such as acetophenone, benzophenone, anthraquinone, anthrone, anthrone-like heterocycles, their substituted derivatives or a combination thereof; and R¹, R², R³, and R⁴ are, independently, spacer elements, which can be null, alkyl or aryl, including, but not limited to cyclic, linear or branched, saturated or unsaturated, aromatic or heteroaromatic, or a combination thereof; and R⁵ and R⁶ are, independently substituents that can be hydrogen, alkyl or aryl, including, but not limited to cyclic, linear or branched, saturated or unsaturated, aromatic or heteroaromatic, or a combination thereof.

More specific examples of linkers with degradable peptide bonds are shown in formula IV, below, wherein R¹ and R² are, independently, substituents that can be hydrogen, alkyl or aryl, including, but not limited to cyclic, linear or branched, saturated or unsaturated, aromatic or heteroaromatic, or a combination thereof; and R³ and R⁴ are, independently, spacer elements, which can be null, alkyl or aryl, including, but not limited to cyclic, linear or branched, saturated or unsaturated, aromatic or heteroaromatic, or a combination thereof

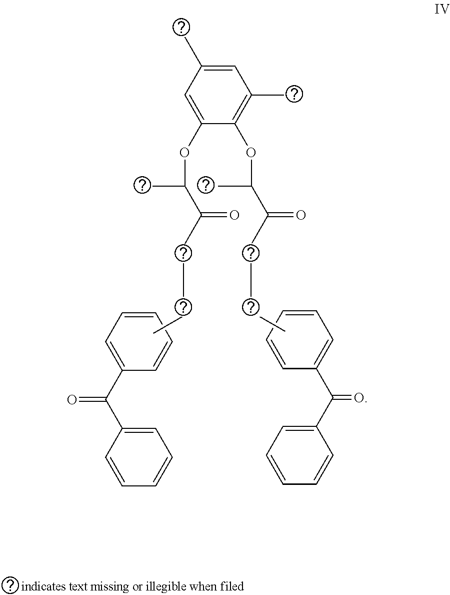

In another embodiment, the degradable linking agent includes one or more ester bonds. Esters can be hydrolyzed to the parent carboxylic acid and an alcohol under acidic or basic conditions. An example of a linker with a degradable ester bond is shown in formula V and VI.

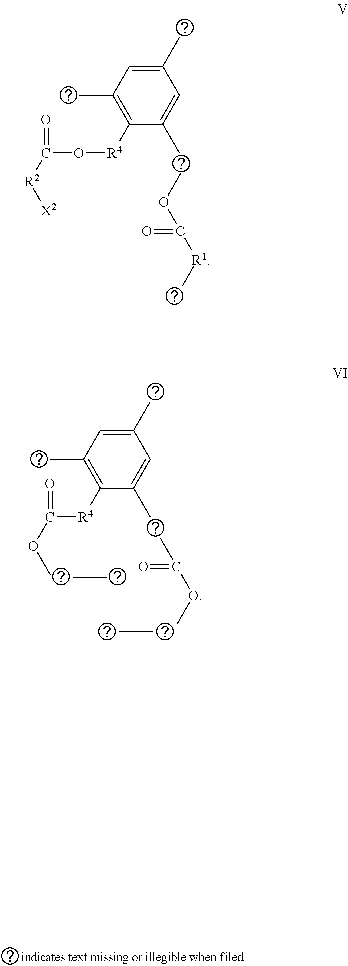

wherein X¹ and X² include, independently, one or more photoreactive groups, including but not limited to aryl ketone photoreactive groups, such as acetophenone, benzophenone, anthraquinone, anthrone, anthrone-like heterocycles, their substituted derivatives or a combination thereof; and R¹, R², are, independently, spacer elements, which can be null, alkyl or aryl, including, but not limited to cyclic, linear or branched, saturated or unsaturated, aromatic or heteroaromatic, or a combination thereof. R³ and R⁴ are, independently, spacer elements, which can be null, a heteroatom, including, but not limited to O, N or S, alkyl or aryl, including, but not limited to cyclic, linear or branched, saturated or unsaturated, aromatic or heteroaromatic, or a combination thereof.

In another embodiment, the degradable linking agent includes one or more thiocarbamate bonds. Thiocarbamates are carbamates in which the C═O group has been replaced by a C═S group. One example of a degradable linker with a thiocarbamate bond can be represented by formula VII:

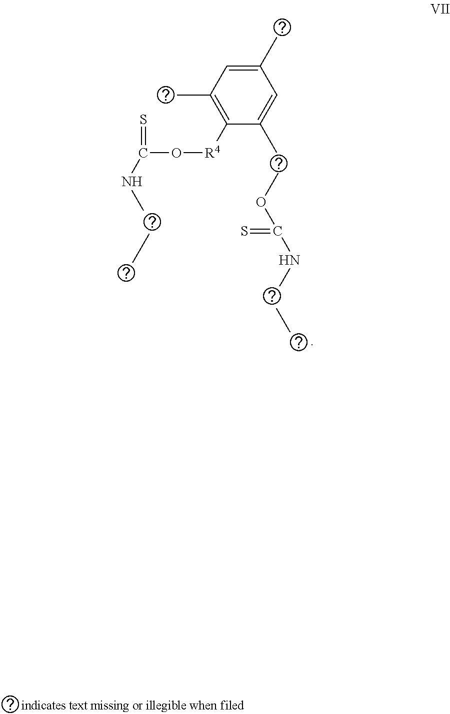

wherein X¹ and X² include, independently, one or more photoreactive groups, including but not limited to aryl ketone photoreactive groups, such as acetophenone, benzophenone, anthraquinone, anthrone, anthrone-like heterocycles, their substituted derivatives or a combination thereof; R¹ and R² are, independently, spacer elements, which can be null, a heteroatom, including, but not limited to O, N or S, alkyl or aryl, including, but not limited to cyclic, linear or branched, saturated or unsaturated, aromatic or heteroaromatic, or a combination thereof; and R³ and R⁴ are, independently, spacer elements, which can be null, alkyl or aryl, including, but not limited to cyclic, linear or branched, saturated or unsaturated, aromatic or heteroaromatic, or a combination thereof.

### Example

### Poly(vinyl difluoride) (PVDF) Membranes Impregnated with Perfluorinated Liquids

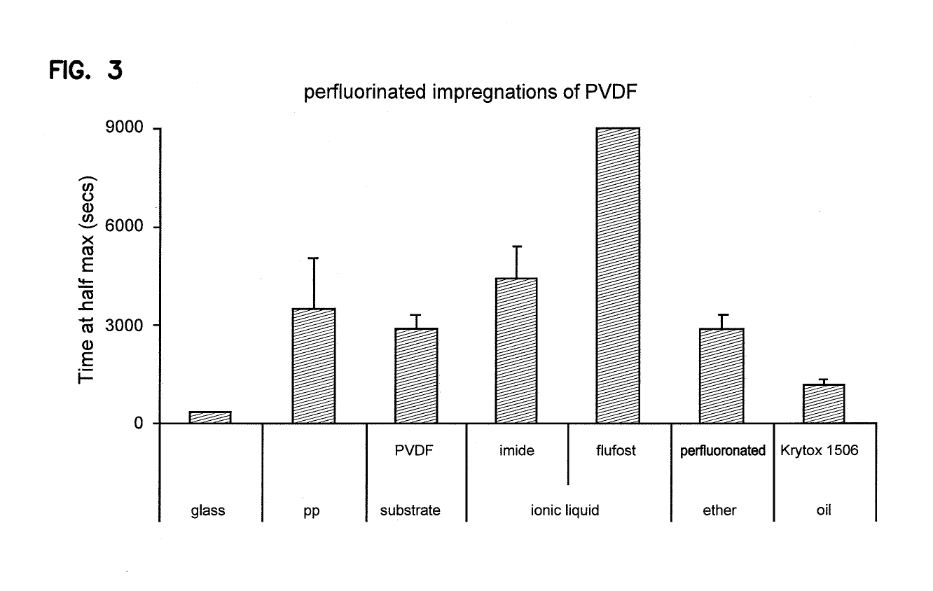

For Examples 1-7 glass, poly(propylene) plate and PVDF syringe filters (available from Cole-Parmer, Vernon Holls, Ill.; cut into 3×5 mm pieces), treated as described in Table 1 below, were placed in a 96-deepwell plate. All samples were exposed to decalcified plasma with cephalin for 20 minutes at 37° C. The ensuing plasma was separated from the solid and transferred to a new plate wherein 55 mM CaCl₂ was added to each well and placed in a plate reader at 37° C. Clotting time was measured using a standard Partial Thromboplastin Time (PTT;) test with absorbance measurements taken at 340 nm every 35 sec over 2.5 hours. FIG. 3. illustrates results for Examples 1-7 of the time at ½ max clotting time.

| | Treatment | Substrate |
|---|---|---|
| Example 1 | None | Glass test tube |
| Example 2 | None | Poly(propylene) (PP) |
| Example 3 | None | PVDF |
| Example 4 | 1-butyl-1-methyl pyrrolidinium | PVDF |
| | bis(trifluoromethylsulfonyl)imide | |
| | (“imide”) | |
| Example 5 | 1-butyl-3-methylimidazolium | PVDF |
| | hexafluorophosphate (“fluoro sf”) | |
| Example 6 | glycidyl dodecafluoroheptyl | PVDF |
| | ether (“ether”) | |
| Example 7 | KRYTOX ™ 1506 | PVDF |

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

We claim:

1. A medical device comprising a superhydrophobic surface or coating, a superoleophobic surface or coating, a coating or surface that is both superhydrophobic and superoleophobic, or a combination of such coatings and surfaces.

2. The medical device of claim 1, wherein the medical device is or comprises an electrophysiology catheter; a self-expanding stent delivery system; a braided metal implant; a flow diverter; a neurological stent; a multi electrode electrophysiology mapping and ablation device; a knitted polymer filament mesh device, e.g., for hernia repair; a urogyncologic sling, a prolapse device; a cosmetic surgery mesh; or a device made of a noble metal.

3. The medical device of claim 1, wherein a portion of the medical device is at least partially coated with or made from a substance that is superhydrophobic, superoleophobic, or both; the portion of the medical device being or comprising: a luminal surface of a coronary stent; a luminal surface of a percutaneous valve delivery catheter; a distal luminal surface where a preloaded implant is in contact with the delivery catheter; a luminal surface of an angiographic or infusion catheter; a fixation pins for a fixation device; an articulated surface of a joint implant; a lumen of a self-expanding stent delivery system; a surface or surface of a self-expanding stent delivery system; an abdominal aortic aneurysm delivery system; an AAA graft; a septal defect device; or a mesh contacting an angiography catheter.

4. The medical device of claim 1 as illustrated in FIG. 1.

5. The medical device of claim 1 as illustrated in FIG. 2.

6. The medical device of claim 1, further comprising a contact portion (29) wherein the contact portion (29) is at least partially coated with a substance selected from the group that is superhydrophobic, superoleophobic or both.

7. The medical device of claim 6, wherein the superhydrophobic group is a slippery liquid-infused porous surface (SLIPS).

8. The medical device of claim 7, wherein the slippery liquid-infused porous surface further comprises 1-butyl-3-methylimidazolium hexafluorophosphate.

9. The medical device of claim 1, further comprising a contact member (25) wherein the contact member (25) is at least partially coated with a substance selected from the group that is superhydrophobic, superoleophobic or both.

10. The medical device of claim 9, wherein the superhydrophobic group is a slippery liquid-infused porous surface (SLIPS).

11. The medical device of claim 11, wherein the slippery liquid-infused porous surface further comprises 1-butyl-3-methylimidazolium hexafluorophosphate.

12. The medical device of claim 1, wherein a portion of the medical device is at least partially coated with or made from a substance that is superhydrophobic, superoleophobic, or both; the portion of the medical device being or comprising: a luminal surface of a coronary stent; a luminal surface of a percutaneous valve delivery catheter; or a surface or surface of a self-expanding stent delivery system.
